(19) United States
(12) Patent Application Publication
(10) Pub. No.: US 2004/0122500 A1
(43) Pub. Date: Jun. 24, 2004

(54) Electrode for utilizing edge effect to create uniform current density

(75) Inventor: Cameron G. Rouns, South Jordan, UT (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/326,851

(22) Filed: Dec. 19, 2002

(51) Int. Cl. A61N 1/05

(52) U.S. Cl. 607/129

(57) ABSTRACT

An electrode for use in stimulating an individual is provided. The electrode includes a conductive element that is at least partially made of a conductive material. The conductive element has an outer edge and has at least one aperture within the outer edge.

### BACKGROUND

Heart fibrillation may be defined as the rapid and uncoordinated contracting of the heart muscle which prevents blood from being properly circulated through the body. This condition may be present in a body that has been subjected to a high degree of electricity, such as when a body contacts a high voltage power line. Additionally, cardiac arrest and trauma to the body, such as an automobile accident, may also result in heart fibrillation. External defibrillators are well known in the art as being devices that are capable of restoring the heart beat to a normal pace through the application of an electrical shock to the body. As such, defibrillators are commonly used in resuscitating patients.

Monitoring of a patient's heart condition and/or defibrillation is often carried out by a physician or paramedic, and requires the physician or paramedic to place one or more monitoring electrodes to the chest of the patient. These monitoring electrodes are then connected to a monitor or defibrillator. If the physician or paramedic must perform the procedure of defibrillation, a pulse of energy needs to be applied to the patient in order to stimulate the patient's heart. The energy may be applied to the patient through the use of paddles that are placed on the body of the patient.

Monitoring devices may include such instruments as cardioscopes, electrocardiographs, and electrocardiograms. These instruments may be used in monitoring the functioning of the heart in addition to monitoring respiration of the patient.

A monitoring device is capable of receiving impulses transferred through the electrode in order to monitor electrical impulses that are made by the patient's heartbeat. These pulses may be displayed on a screen of the monitoring device for analysis by a physician or paramedic.

A monitoring device or defibrillating device may also be used to provide an electrical impulse to the patient's heart. This electrical impulse can be a regularly timed impulse that is used to “pace” the heartbeat of a patient through regular, consistent pulses. Additionally, the monitoring or defibrillating device may provide a very strong electrical impulse to the patient in order to quickly stimulate the heart.

Therapeutic devices are also devices that may employ electrodes. Therapeutic devices include electro-surgical units and radio frequency applicators. These devices may be used to apply electrical energy to a patient in order to reduce pain and to promote healing of injuries.

All of these instruments commonly employ electrodes, which are small conducting plates. These conducting plates allow for the transmission of electrical impulses to and from the patient. Electrical conductivity between the electrodes and the patient's skin is usually completed by means of a saline gel that is applied to the surface of the electrode and contacts the patient's skin. An electrical lead is often placed from the conducting plate of the electrode to the instrument onto which it is attached, that is the specific monitoring device, stimulating device, and/or therapeutic device. The electrodes themselves are typically considered disposable objects so that they are discarded after use. The electrical leads to the electrodes are often used again. Prior electrodes make use of snap-on connections in order to attach and disattach the leads therefrom.

It is often the case that the electrodes are placed on the chest of the patient being resuscitated or being monitored. It is sometimes the case that better results in defibrillating a patient are achieved when one of the defibrillating electrodes is placed on the front of the patient and the other is placed on the back of the patient. This type of arrangement is thought to provide an increased amount of current to the heart and thus increase the chances of a successful resuscitation of the patient.

Current electrodes suffer from a problem commonly known as “edge effect”. Edge effect occurs when the electrical charge that is conducted through the electrode becomes concentrated at the outer edges of the electrode, as opposed to being spread uniformly throughout the entire surface of the electrode. By having a high concentration of electrical current at the outer edges of the electrode, this electrical current results in the burning of the patient.

In order to overcome the edge effect, prior electrodes have been designed such that a second conductive layer is placed in contact with the outer edges of the electrode. In this case, the high current at the outer edges of the electrode will be transferred into the second conductive plate and more evenly spread therefrom. Both the electrode and the second conducting plate have a hydrogel applied thereon which permits electrical conductivity from the electrode and the second conducting plate into the patient.

The present invention provides for an improved electrode that utilizes the edge effect to impart a more advantageous distribution of current in order to stimulate a patient.

### SUMMARY

Various features and advantages of the invention will be set forth in part in the following description, or may be obvious from the description, or may be learned from practice of the invention.

The present invention provides for an electrode for monitoring and/or transmitting energy to an individual. The electrode includes a conductive element that is at least partially made of a conductive material. The conductive element has an outer edge and at least one aperture on the outer edge defining an inner edge of the conductive element.

In other exemplary embodiments of the present invention, the electrode may have a flat conductive element. Further, an electrical lead may be connected to the conductive element and be in electrical communication with the conductive element. A hydrogel layer may be incorporated onto the conductive element and allow for electrical communication between the conductive element and the patient through contact of the skin of the patient. A further exemplary embodiment of the present invention exists where the electrode has a foam backing that engages the conductive element.

The present invention includes various exemplary embodiments where the aperture or apertures assume various shapes and sizes. For instance, in one exemplary embodiment of the present invention the apertures are arc shaped, while in another exemplary embodiment of the present invention the apertures are circular holes. Further, an exemplary embodiment of the present invention exists where the apertures are substantially straight sections that are substantially parallel to one another. Any number of apertures may be provided. For instance in one exemplary embodiment of the present invention, five apertures are provided and may be either arc shaped or substantially straight sections that are parallel to one another.

As stated, the plurality of apertures may assume any number, size, and shape. In one exemplary embodiment of the present invention the plurality of apertures includes a first pair of openings that are located near an end of the conductive element and extend through the conductive element. Each of the first pair of openings has a substantially straight section and an arc shaped section that is contiguous with the substantially straight section. A second pair of openings are also provided and are located adjacent to the first pair of openings. Each of the second pair of openings has three substantially straight sections where one of the sections is at a substantially right angle to and contiguous with the other two sections. Also, a third pair of openings are provided. The third pair of openings are substantially straight and each of the third pair of openings are substantially parallel with two of the straight sections of the second pair of openings. Also, each of the third pair of openings is located between two of the straight sections of the second pair of openings. Further, a fourth pair of openings are provided and are substantially straight and are located adjacent to the second pair of openings. Each of the fourth pair of openings are substantially parallel to two of the substantially straight sections of the second pair of openings. A fifth opening is located adjacent to the fourth pair of openings and is substantially T-shaped. Finally, a sixth pair of openings are located near an end of the conductive element and are adjacent to the fifth opening. Each one of the sixth pair of openings is arc shaped.

An additional exemplary embodiment includes an electrode that has a conductive element with at least one recess that does not extend all the way through the conductive element.

Although described as having apertures, the conductive element may instead, or in addition have at least one protrusion located thereon for forming the inner edge in other exemplary embodiments.

### BRIEF DESCRIPTION OF THE DRAWINGS

FIG. 1 is a plan view of an exemplary embodiment an electrode in accordance with the present invention.

FIG. 1a is a cross sectional view taken along line 1a of FIG. 1. The view shows a hydrogel layer being disposed on a foam backing and a conductive element.

FIG. 2 is an exploded assembly view of an exemplary embodiment of an electrode in accordance with the present invention.

FIG. 3 is a perspective view of an individual having a pair of electrodes applied thereto.

FIG. 4 is a perspective view of an exemplary embodiment of a conductive element in accordance with the present invention. Here, a plurality of apertures being holes are disposed on the conductive element.

FIG. 5 is a perspective view of an exemplary embodiment of a conductive element in accordance with the present invention. Here a plurality of apertures are disposed on the surface of the conductive element.

FIG. 6 is a perspective view of a conductive element in accordance with another exemplary embodiment of the present invention. Here, a series of arc shaped openings are disposed on the surface of the conductive element.

FIG. 7 is a perspective view of a further exemplary embodiment of a conductive element in accordance with the present invention. Here, a plurality of substantially straight sections are disposed on the surface of the conductive element.

FIG. 8 is a perspective view of a conductive element in accordance with an exemplary embodiment of the present invention. Here, a plurality of partially extending recesses are present on the upper surface of the conductive element.

FIG. 9 is a perspective view of a conductive element in accordance with the present invention. Here, a series of protrusions are disposed on the conductive element.

### DETAILED DESCRIPTION

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations. It is commonly believed that with a finite amount of current traveling through a conductor of fixed area, any method of design or delivery that contributes to a uniform delivery of such energy has a beneficial effect of reducing the occurrence of skin effects and burns.

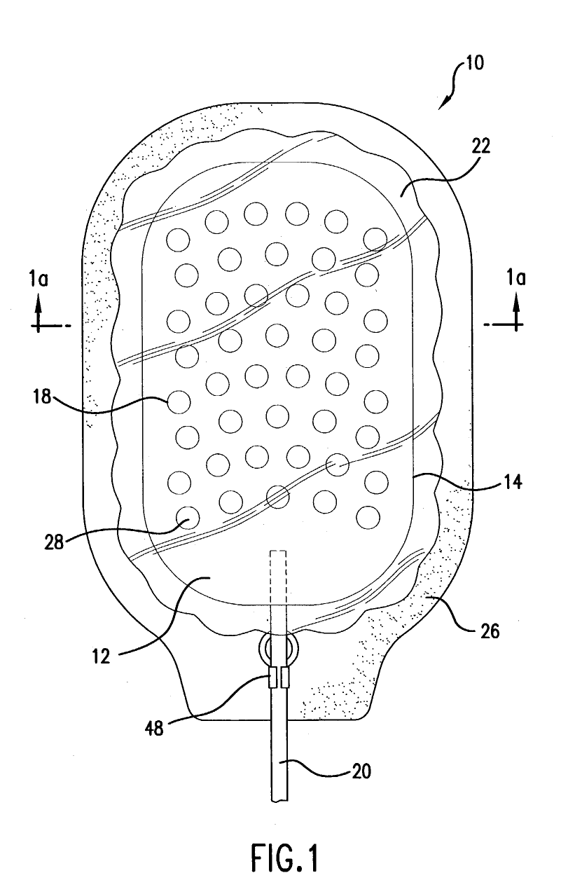

Referring now to the drawings, FIG. 1 shows an electrode 10 in accordance with one exemplary embodiment of the present invention. The electrode 10 is shown as being composed of a foam backing 26 that houses a conductive element 12. The conductive element 12 may be secured to the foam backing 26 through a variety of means. For instance, the foam backing 26 may be formed around the conductive element 12 in one exemplary embodiment of the present invention. Alternatively, the conductive element 12 may be adhered to the foam backing 26 through adhesion or other means such as bolts or pins.

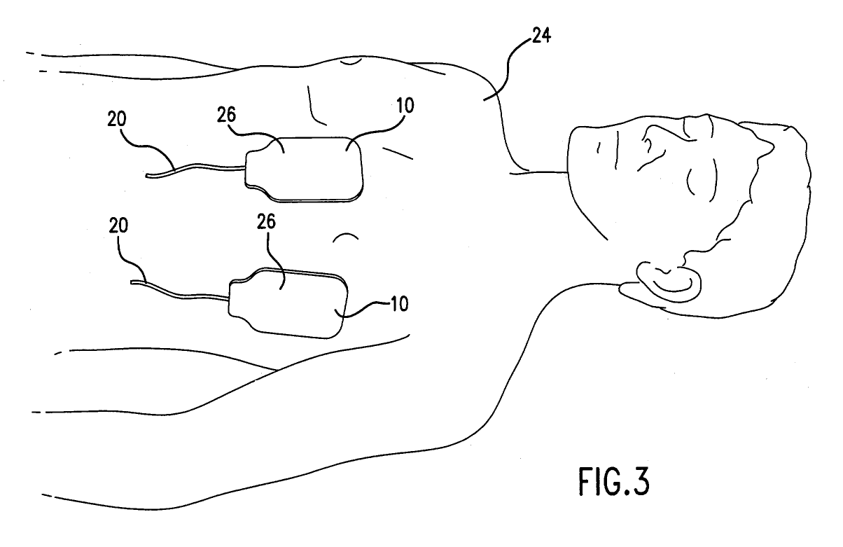

Although the bottom portion of the electrode 10 is shown in FIG. 1, the top portion of the exemplary embodiment shown in FIG. 1 (which is visible in FIG. 3) may have some, all, or none of the conductive element 12 being visible. All references to the “top” and “bottom” are for purposes of explanation of the disclosure and are not meant to limit the invention. For instance, one may refer to the “bottom” as the “top” and the “top” as the “bottom”. These directional words are not meant to define the invention, but only to more clearly explain the disclosure. The top portion may be thought of as the side of the electrode 10 facing the clinician when the electrode 10 is applied to an individual 24. As such, the foam backing 26 may extend across all, some, or none of the conductive element 12. However, in one exemplary embodiment of the present invention it is advantageous to have the foam backing 26 extend across all of the top surface of the conductive element 12 because doing so will prevent inadvertent electrical discharge to a clinician. However, the present invention is not limited to having the foam backing 26 extend across the entire top surface of the conductive element 12. FIG. 3 shows an exemplary embodiment of the present invention where the foam backing 26 does extend across the entire top surface of the conductive element 12. As such, FIG. 3 shows a pair of electrodes 10 wherein the conductive element 12 is not visible and contacts the skin of an individual 24, although in some instances hydrogel may be disposed between the conductive element 12 and the skin. The foam backing 26 may be a material that inhibits the transfer of electrical current therethrough. As such, a clinician may contact the foam backing 26 without being shocked by current present in the conductive element 12.

As stated, the conductive element 12 may be any type of material that allows the transfer of electrical current therethrough. For instance, in one exemplary embodiment of the present invention the conductive element 12 may be made of aluminum. In other exemplary embodiments, the conductive element 12 may be made of copper, carbon, or steel. Additional exemplary embodiments of the present invention include any conductive material comprising the conductive element 12. Typically, the conductive element 12 is a thin sheet. However, in other embodiments the conductive element 12 is thicker and exhibits more rigidity. The conductive element 12 of the present invention is not limited to a particular thickness, shape, or material. Although shown as being rectangular in shape, the conductive element 12 may be of any shape, and is not limited to a rectangular configuration.

As seen in FIG. 1, the conductive element 12 may be substantially rectangular shaped having corners that are rounded. An outer edge 14 of the conductive element 12 abuts against the foam backing 26. A plurality of apertures are present in the conductive element 12 shown in FIG. 1. The apertures are circular holes 28 which are present across a substantial portion of the surface of the conductive element 12. Although the holes 28 are shown as being identical in shape and size, in one exemplary embodiment of the present invention the holes 28 may be of varying sizes and/or shapes. The holes 28 form an inner edge 18 on the conductive element 12.

Electrical current is supplied to the conductive element 12 through an electrical lead 20. The electrical lead 20 is connected to the foam backing 26 by a connection member 48. Electrical current and/or data may be transmitted or communicated to the conductive element 12 through the electrical lead 20. The electrical lead 20 may further be connected to surgical equipment (not shown) that may monitor, defibrillate, and/or provide therapeutic treatment or the like via electrical impulses to the individual 24. Also, data or electrical energy may be transmitted from the conductive element 12 through the electrical lead 20 to the piece of surgical equipment (not shown). As such, the electrode 10 provides for communication and transfer of energy and/or data to and from an individual 24.

As stated, electrical current transmitted to an individual 24 from an electrode 10 may burn the individual 24 due to an edge effect that is present on a conductive element 12. When electrical current is passed through the conductive element 12 there is an increase in current flow at the periphery of the conductive element 12. In effect there is greater current at the outer edge 14 of the conductive element 12 than in the middle of the conductive element 12. Again, this phenomenon is known as “edge effect”. The conductive element 12 disclosed uses the edge effect to provide a more uniform pattern of current density during the process of providing electrical energy to the individual 24 through the conductive element 12. The presence of the apertures, which are shown as holes 28 in FIG. 1, increases both the number and length of edges that are present and reduces the current density due to the fact that a finite amount of current is present. This increase in the edges of the conductive element 12 results in fewer areas of high current density and therefore reduces the increased current at the outer edge 14 of the conductive element 12. Also, the presence of the inner edge 18 helps to increase the current flow in the center area of the conductive element 12 which in turn tends to reduce the amount of current at the outer edge 14 of the conductive element 12. Therefore, the presence of the apertures use the edge effect present in the conductive element 12 to disperse energy is intended to significantly reduce the chance of burning the individual 24 during use of the electrode 10. Although shown as being a plurality of apertures in FIG. 1, it is to be understood that the conductive element 12 in accordance with the present invention may have any number of apertures. For instance, one aperture or any number greater than one may be used.

The present invention includes exemplary embodiments where the apertures do not contact the outer edge 14 of the conductive element 12. As such, the apertures may be distanced from the outer edge 14 of the conductive element 12.

FIG. 1a shows a cross sectional view of the electrode 10 taken along line 1a of FIG. 1. Here, a hydrogel layer 22 is present and is shown as being applied to the upper surface of the conductive element 12 and the foam backing 26. Hydrogels 22 are typically used in the application of electrical current to an individual 24 through electrodes 10. Hydrogels 22 are commonly a liquid gel that allows for the conduction of electrical current from the electrode 10 into the individual 24. The hydrogel 22 may be applied to the electrode 10 before the electrode 10 is contacted with the skin of individual 24, or if desired the hydrogel 22 may be placed on the individual 24 before the application of the electrode 10 thereon. The hydrogel 22 is advantageous in that it is typically a tacky substance that may easily adhere to the skin of the individual 24 and also adhere to the electrode 10, helping to secure the electrode 10 thereon. Adhesives may be used around the edges of the electrode 10 to secure it to the individual 24.

Although shown as positioning a pair of electrodes 10 onto the chest of the individual 24, the present invention is not limited to electrodes 10 that are only placed on the chest of the individual 24. For instance, in other exemplary embodiments of the present invention the electrode 10 may be placed on the back, arms, and/or leg of the individual 24. In one exemplary embodiment of the present invention, an electrode 10 may be positioned on the chest of the individual 24 while another electrode 10 is positioned on the back of the individual 24.

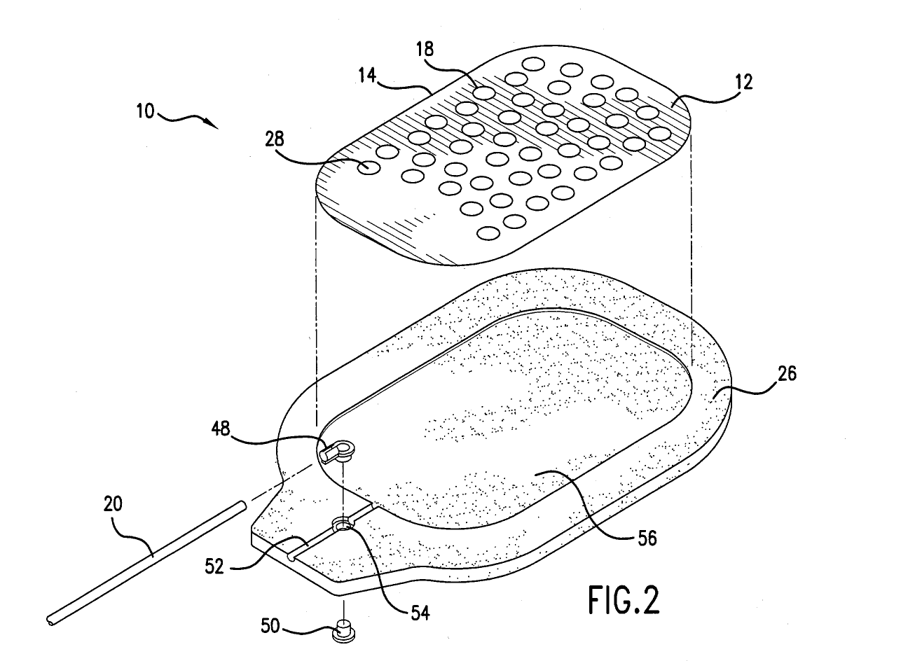

FIG. 2 is an assembly view of one exemplary embodiment of the electrode 10 in accordance with the present invention. Here, the foam backing 26 is provided with a foam backing recess 56 which receives the conductive element 12. The conductive element 12 is substantially similar to the conductive element 12 disclosed in FIG. 1 having a plurality of holes 28 disposed across the upper surface of the conductive element 12. The conductive element 12 may be retained within the foam backing recess 56 through a variety of means, such as adhesion applied between the foam backing 26 and the conductive element 12, sonic welding processes, or by mechanical fasteners such as pins or bolts. Additionally, the conductive element 12 may be integrally formed with the foam backing 26 and therefore retained in the foam backing recess 56. This can be accomplished, for instance, by molding the foam backing 26 around the conductive element 12. Alternatively the foam recess may be created by the use of a second layer of foam applied to the edge of the electrode 10. In another embodiment the foam may sandwich the hydrogel 22 and/or conductive element 12 in part between the foam so as to maintain the positioning of the conductive element 12.

The electrical lead 20 may be attached to the foam backing 26 by use of the connection member 48. A channel 52 is shown as being present within the foam backing 26 and accommodates the insertion, passage, and retention of the electrical lead 20 onto and through the foam backing 26. A connection member recess 54 may be present in the foam backing 26 and accommodates insertion of the connection member 48 therein. The connection member 48 may be affixed to the foam backing 26 by the use of a pin 50. The pin 50 therefore holds the connection member 48 onto the foam backing 26 which in turn holds the electrical lead 20. Although not shown, an insulated material may be applied on top of the pin 50 so that current that is transmitted through the electrical lead 20 into the connection member 48 and finally into the pin 50 and is not transmitted to a clinician or other person who comes in contact with the electrode 10. The electrical lead 20 should be extended through the connection member 48 so that it contacts the conductive element 12. This contact allows for electrical communication to and from the electrical lead 20 and the conductive element 12.

Although the connection of the electrical lead 20 to the foam backing 26 is shown as occurring in the foam backing 26, it is to be understood that other arrangements are possible. For instance, in one exemplary embodiment of the present invention the connection member 48 may be disposed on the conductive element 12. In this case, the connection member recess 54 may be present in the foam backing recess 56 of the foam backing 26. In this instance, the electrical lead 20 need only contact the connection member 48 in order to transmit electrical current to and from the conductive element 12. This is because the connection member 48 will be in physical contact with the conductive element 12 and therefore provide for communication between the conductive element 12 and the electrical lead 20. However, the arrangement may be such that the electrical lead 20 also contacts the conductive element 12. This may be advantageous in that a greater degree of contact and electrical transfer is present.

Although shown as employing the connection member 48, in other exemplary embodiments the use of the connection member 48, pin 50, channel 52, and/or the connection member recess 54 may not be needed. For example, in the case where these parts are not used, the electrical lead 20 may be a copper wire that is in a splay configuration and contacts the conductive element 12 in order to allow for electrical transfer. Any of a variety of suitable connectors between the lead 20 and the conductive element 12 may be used.

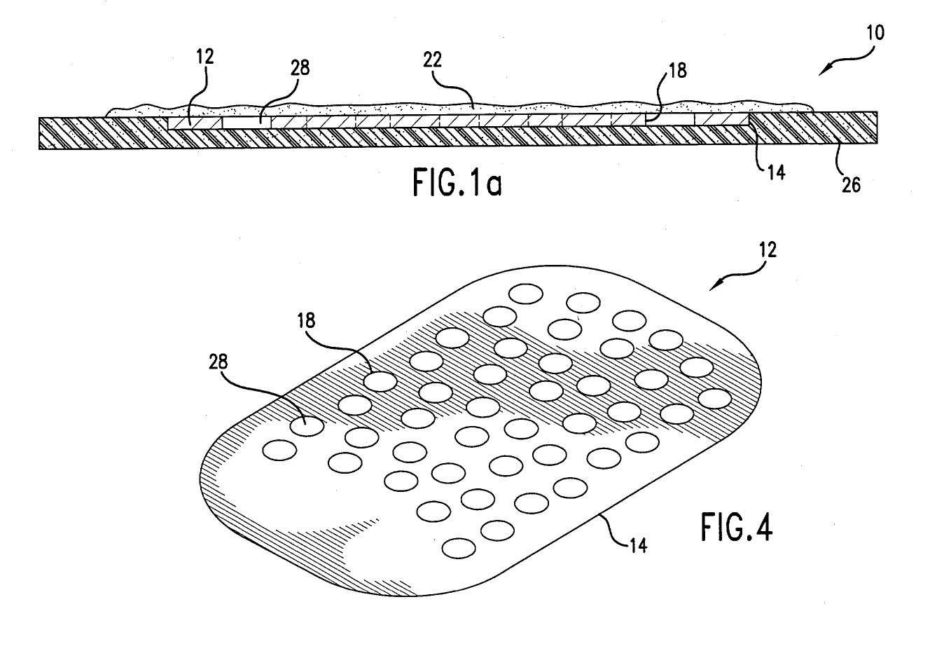

The conductive element 12 according to one exemplary embodiment of the present invention is shown in FIG. 4. Here, the apertures include a plurality of holes 28 that are located in eight rows across the surface of the conductive element 12. Each row includes approximately five holes 28. As shown, an area towards an end of the conductive element 12 may be provided with no holes 28 being present in order to allow for connection of the electrical lead 20 as discussed above. Although, the holes 28 are shown as having the same diameter, it is to be understood that in other exemplary embodiments of the present invention holes 28 having different diameters may be present across the surface of the conductive element 12. Additionally, the apertures may take any shape or form and be present in any size or number in accordance with the present invention. As such, the electrode 10 of the present invention is not limited to a conductive element 12 having only the shapes, sizes, and locations of the apertures as shown in the disclosed figures.

The exemplary embodiment of the conductive element 12 shown in FIG. 4 has 44 holes 28 being present. Each of the holes 28 forms an inner edge 18 that is 0.94″ in circumference (although the drawings are not to scale, including FIG. 4). Multiplying this distance by the number of holes 28 reveals the configuration of the apertures in FIG. 4 provide about 41.47″ of inner edge 18 length in the conductive element 12. The outer edge 14 of the conductive element 12 is 14.59″ in length. Therefore, the total edge present in the conductive element 12 of FIG. 4 is the length of the outer edge 14 (14.59″)+the length of the inner edge 18 (41.47″) which is 56.06″. The % of increase of edge caused by the presence of the holes 28 in FIG. 4 may be defined by the following equation:

$$\% increase of edge = \frac{( total edge length ) - ( length of outer edge 14 )}{( length of outer edge 14 )} \\ = \frac{( 56.06 ) - ( 14.59 )}{( 14.59 )} ≅ 284 \%$$

Therefore, providing the conductive element 12 with the pattern of the holes 28 shown in FIG. 4 results in an increase of approximately 284% in the amount of edge surface that is present in the conductive element 12. This helps to reduce the concentration of current due to edge effect when using the electrode 10.

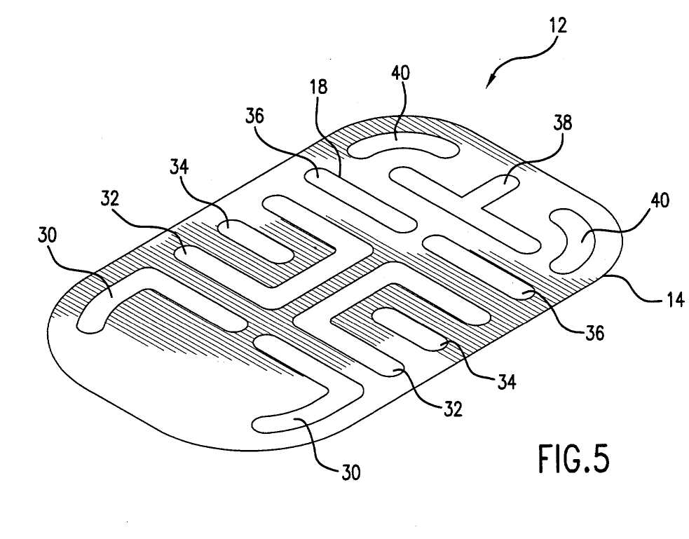

FIG. 5 shows another exemplary embodiment of the conductive element 12 in accordance with the present invention. Here, the apertures are positioned across the face of the conductive element 12 in order to reduce the edge effect that is present proximate to the outer edge 14 during use of the conductive element 12. Again, a portion of the conductive element 12 near one end is not provided with an aperture in order to allow for the connection of the electrical lead 20 as discussed above. However, it is to be understood that providing a portion of the conductive element 12 without an aperture is not necessary in this or other exemplary embodiments.

The apertures disclosed in FIG. 5 include a first pair of openings 30 that are located near an end of the conductive element 12, and proximate to the location of the conductive element 12 that does not have an aperture being present. Each of the first pair of openings 30 includes a substantially straight section and an arc shaped section that is contiguous with the substantially straight section. The arc shaped section of the first pair of openings 30 contacts the substantially straight section at approximately the end of the substantially straight section.

Located next to the first pair of openings 30 are a second pair of openings 32 that are also located on the conductive element 12. Each of the second pair of openings 32 includes three substantially straight sections. One of the substantially straight sections of the second pair of openings 32 is substantially at a right angle to the other two substantially straight sections of the second pair of openings 32. One of the substantially straight sections of the second pair of openings 32 is contiguous with the other two substantially straight sections and contacts them at their ends. A third pair of openings 34 are present and are located between two of the substantially straight sections of the second pair of openings 32. The third pair of openings 34 are also substantially straight.

A fourth pair of openings 36 are present and are substantially straight. The fourth pair of openings 36 are proximate to the second pair of openings 32 and are substantially parallel with two of the substantially straight sections of the second pair of openings 32. A fifth opening 38 is present and is proximate to the fourth pair of openings 36 and also proximate to an end of the conductive element 12. The fifth opening is roughly “T” shaped. A sixth pair of openings 40 are proximate to the fifth opening 38 and also proximate to an end of the conductive element 12. The sixth pair of openings are arc shaped. The pattern of the apertures disclosed in FIG. 5 provide for a reduction in the edge effect present on the conductive element 12 due to a substantially increased amount of the inner edge 18 being present on the conductive element 12.

The conductive element 12 of FIG. 5 has the following apertures and inner edges 18:

| Apertures | Inner edge 18 |
|---|---|
| First Pair of Openings 30 | 4.51″ each |
| Second Pair of Openings 32 | 6.58″ each |
| Third Pair of Openings 34 | 1.78″ each |
| Fourth Pair of Openings 36 | 2.78″ each |
| Fifth Opening 38 | 4.89″ |
| Sixth Pair of Openings 40 | 2.89″ each |
| | Total 42.02″ |

The total edge being the outer edge 14 plus the total of the inner edges 18. The outer edge 14 is 14.59″ in length, and the total edge is therefore 14.59″+42.02″=56.61″. The % increase of edge is:

$$\% increase of edge = \frac{( 56.61 ) - ( 14.59 )}{( 14.59 )} = 288 \%$$

As can be seen, using the pattern of the apertures on the conductive element 12 in FIG. 5 results in about a 288% increase of edge. This increase in edge is believed to reduce burning due to the edge effect.

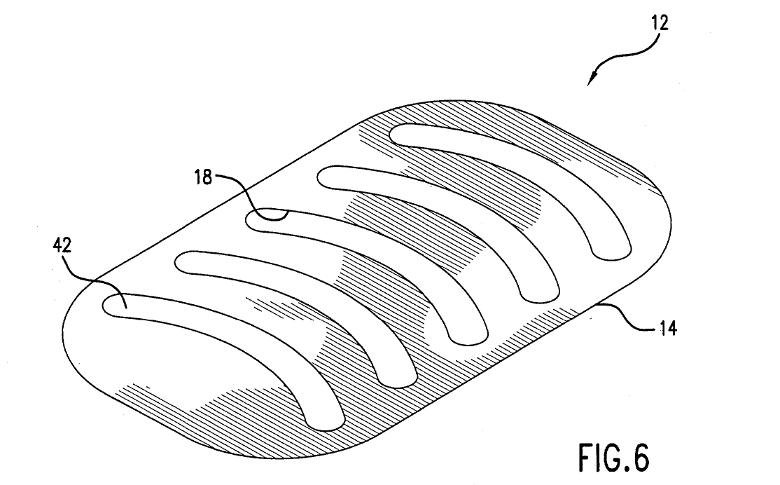

FIG. 6 shows another exemplary embodiment of the conductive element 12 in accordance with the present invention. Here, five apertures are present on the surface of the conductive element 12. Each of the apertures are an arc shaped opening 42. The arc shaped openings 42 extend from approximately one side of the conductive element 12 to the other, having the high point of the arc being approximately half way between the respective sides of the conductive element 12. The arc shaped openings 42 are oriented in one direction and are evenly spaced across the surface of the conductive element 12. Again, the orientation of the arc shaped openings 42 allows for a certain portion of the conductive element 12 to be free of an aperture in order to allow for the connection of the electrical lead 20 as discussed above. Of course, in other exemplary embodiments of the present invention, the arc shaped openings 42 may be oriented differently so that the arc of the arc shaped opening 42 is not at the mid point between two of the sides of the conductive element 12. Additionally, greater or fewer than five of the arc shaped openings 42 may be present in other exemplary embodiments.

Each of the arc shaped openings 42 in the exemplary embodiment of the conductive element 12 shown in FIG. 6 forms an inner edge 18 length that is 5.92″ in length. The outer edge 14 is 14.59″ in length. Therefore, the total of the inner edge 18 length is (5) X (5.92″)=29.62″. The total edge of the conductive element 12 will therefore be the outer edge 14+the total of the inner edge 18, which is (14.59″)+(29.62″)=44.21″. The % of increase of edge caused by the addition of the arc shaped openings 42 is determined by the following equation:

$$\% increase of edge = \frac{( {44.21}^{″} ) - ( {14.59}^{″} )}{( {14.59}^{″} )} = 203 \%$$

As can be seen, the addition of the five arc shaped openings 42 causes about a 203% increase in the amount of edge that is present on the conductive element 12. Again, this increase in total edge will help to reduce the concentration of current due to the edge effect when using the electrode 10.

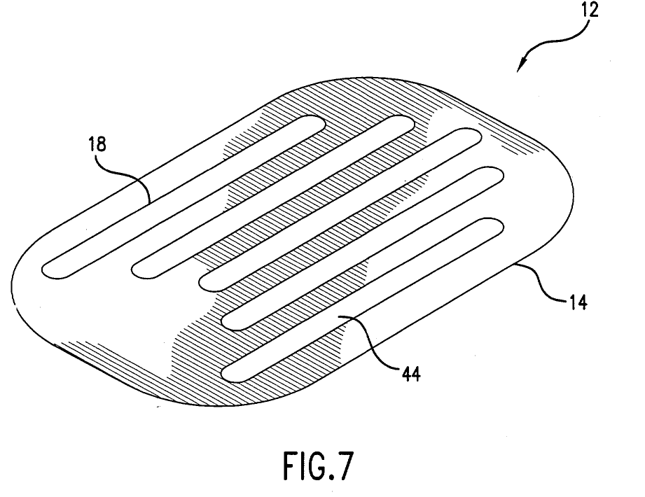

FIG. 7 shows an exemplary embodiment of the conductive element 12 in accordance with the present invention. Here, the apertures are a series of substantially straight sections 44. Each of these substantially straight sections 44 may be substantially parallel with one another. Six of the substantially straight sections 44 are provided across the surface of the conductive element 12. Again, a portion of the conductive element 12 is not provided with an aperture in order to allow for the connection of the electrical lead 20 as discussed in previous embodiments.

In FIG. 7 the conductive element 12 has five substantially straight sections 44 that each form an inner edge 18 that is 6.94″ in length. The outer edge 14 of the conductive element 12 is 14.59″ in length. Therefore, the total edge length is (6)×(6.94″)+14.59″=49.30″. The % of increase of edge due to the presence of the five substantially straight sections 44 maybe calculated by the following equation:

$$\% increase of edge = \frac{( {49.30}^{″} ) - ( {14.59}^{″} )}{( {14.59}^{″} )} = 238 \%$$

As can be seen, the presence of the five substantially straight sections 44 on the conductive element 12 causes about a 238% increase in the amount of edge present. Again, this increase in the amount of edge will result in a reduced edge effect upon use of the electrode 10.

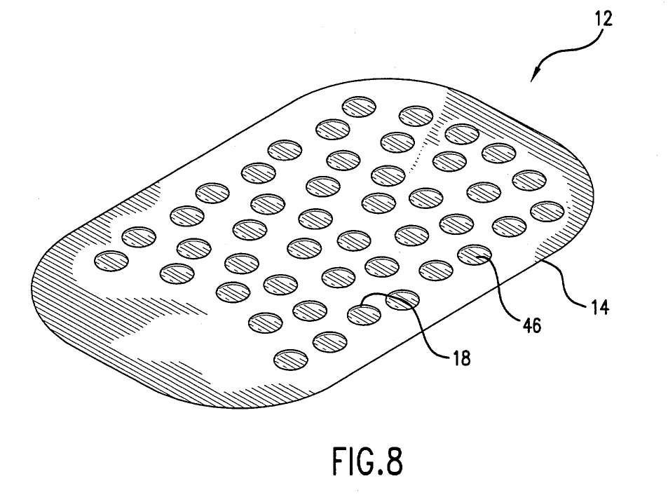

FIG. 8 shows another exemplary embodiment of the present invention where instead of apertures, at least one recess 46 which does not go all the way through the conductive element 12 is present. The recess 46 may extend halfway through the thickness of the conductive element 12, or may extend through any portion of the conductive element 12 in other exemplary embodiments of the present invention. Similar to those embodiments having apertures, the inner edges 18 of the conductive element 12 work in a similar way to reduce the edge effect upon use of the electrode 10. The inner edges 18 are present on the recesses 46 and reduce the concentration of electrical current at the outer edge 14 of the conductive element 12.

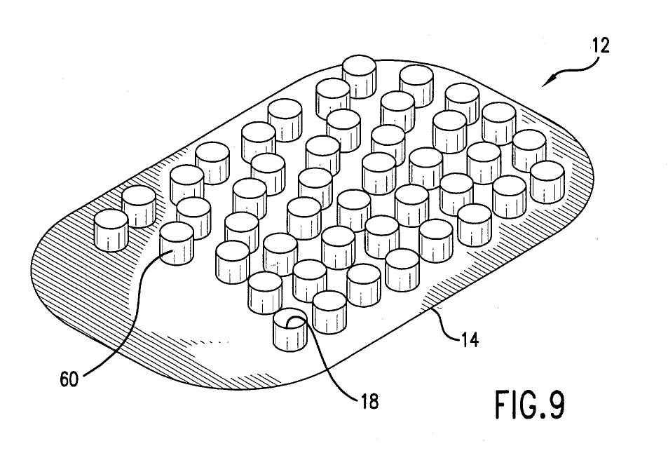

Although described as having apertures, it is to be understood that other exemplary embodiments of the present invention may be provided with at least one protrusion 60 as shown in FIG. 9. The protrusion 60 is capable of forming the inner edge 18 in much the same way as the apertures as previously described. The inner edge 18 in FIG. 9 works to reduce the “edge effect” in much the same way as in the previous exemplary embodiments of the present invention. The protrusions 60 may be provided in various numbers, sizes, and shapes in accordance with the present invention. Additionally, a combination of the protrusions 60, recesses 46, and the apertures may be provided in other exemplary embodiments. The protrusion 60 may be the same material as the conductive element 12, or may be made from a different material. Additionally, the protrusions 60 may be formed on one or both sides of the conductive element 12, and may face or face away from the individual 24 during use.

The present invention is not limited to a particular % or a particular range of % of edge increase due to the apertures. Also, the present invention is not limited to the disclosed lengths of the inner edges 18 and outer edge 14. Any sized conductive element 12 may be used.

The present invention therefore includes exemplary embodiments where the % of increase of edge due to the presence of the apertures, is greater than 200%. More specifically, the present invention provides for exemplary embodiments where the increase in edge due to the presence of the recess 46, protrusions 60 and/or apertures is between 200% and 300%. However, it is to be understood that in other exemplary embodiments of the present invention that the % of increase of edge may be greater than 300% or less than 200%. For instance, in certain exemplary embodiments of the present invention, the % of increase of edge may be as low as 1% or any increase greater than 0% due to the presence of one or more apertures.

It is to be understood that the present invention includes various modifications that can be made to the embodiments of the electrode 10 described herein as come within the scope of the appended claims and their equivalents.

What is claimed is:

1. An electrode comprising:
a conductive element being at least partially made of a conductive material, said conductive element having an outer edge, said conductive element having at least one aperture within said outer edge defining at least one distinct inner edge of said conductive element.

2. The electrode of claim 1, further comprising an electrical lead connected to said conductive element and being in electrical communication with said conductive element.

3. The electrode of claim 1, further comprising a hydrogel layer contacting said conductive element and being in electrical communication with said conductive element, said hydrogel layer configured to allow electrical communication between said conductive element and the individual.

4. The electrode of claim 1, wherein said conductive element is made of a material selected from the group consisting of aluminum, steel, cooper, and carbon.

5. The electrode of claim 1, further comprising a foam backing engaging said conductive element.

6. The electrode of claim 1, wherein said apertures being a plurality of apertures extending through said conductive element.

7. The electrode of claim 6, wherein said plurality of apertures comprising:
a first pair of openings located near an end of said conductive element and extending through said conductive element, each of said first pair of openings having a substantially straight section and an arc shaped section contiguous with said substantially straight section;
a second pair of openings located adjacent to said first pair of openings, each of said second pair of openings having three substantially straight sections wherein one of said three substantially straight sections being at a substantially right angle to and contiguous with the other two of said three substantially straight section;
a third pair of openings, said third pair of openings being substantially straight and each of said third pair of openings being substantially parallel with two of said straight sections of said second pair of openings, and each of said third pair of openings located between two of said straight sections of said second pair of openings;
a fourth pair of openings being substantially straight and being located adjacent to said second pair of openings, each of said fourth pair of openings being substantially parallel to two of said substantially straight sections of said second pair of openings;
a fifth opening located adjacent to said fourth pair of openings, said fifth opening being substantially T-shaped; and
and a sixth pair of openings located near an end of said conductive element and adjacent to said fifth opening, each of said sixth pair of openings being arc shaped.

8. The electrode of claim 6, wherein at least one of said plurality of apertures being arc shaped.

9. The electrode of claim 6, wherein each of said apertures is a substantially straight section, and wherein each of said substantially straight sections is substantially parallel to one another.

10. The electrode of claim 1, wherein;
a total edge length of said conductive element being defined as the length of said outer edge plus the length of said inner edge; and
wherein the addition of said at least one aperture to said conductive element causing a percentage increase of edge of greater than about 200% where said percentage of increase of edge being defined by the equation:
$$\% increase of edge = \frac{( total edge length ) - ( length of outer edge )}{( length of outer edge )} .$$

11. The electrode of claim 10, wherein said percentage of increase of edge being between about 200% and about 300%.

12. An electrode comprising:
a conductive element being at least partially made of a conductive metal, said conductive element having an outer edge, said conductive element having at least one aperture within said outer edge defining at least one distinct inner edge of said conductive element;
an electrical lead connected to said conductive element and being in electrical communication with said conductive element; and
a hydrogel layer contacting said conductive element and being in electrical communication with said conductive element, said hydrogel layer configured to allow electrical communication between said conductive element and an individual.

13. The electrode of claim 12, wherein said conductive element is made of a material selected from the group consisting of aluminum, copper, steel, and carbon.

14. The electrode of claim 12, further comprising a foam backing engaging said conductive element.

15. The electrode of claim 12, wherein said aperture being a plurality of apertures extending through said conductive element.

16. The electrode of claim 15, wherein said plurality of apertures comprising:
a first pair of openings located near an end of said conductive element and extending through said conductive element, each of said first pair of openings having a substantially straight section and an arc shaped section contiguous with said substantially straight section;
a second pair of openings located adjacent to said first pair of openings, each of said second pair of openings having three substantially straight sections wherein one of said three substantially straight sections being at a right angle to and contiguous with the other two of said three substantially straight sections;
a third pair of openings, said third pair of openings being substantially straight and each of said third pair of openings being substantially parallel with two of said straight sections of said second pair of openings, and each of said third pair of openings located between two of said straight sections of said second pair of openings;
a fourth pair of openings being substantially straight and being located adjacent to said second pair of openings, each of said fourth pair of openings being substantially parallel to two of said substantially straight sections of said second pair of openings;
a fifth opening located adjacent to said fourth pair of openings, said fifth opening being substantially T-shaped; and
a sixth pair of openings located near an end of said conductive element and adjacent to said fifth opening, each of said sixth pair of openings being arc shaped.

17. The electrode of claim 15, wherein each of said plurality of apertures being arc shaped, and said plurality of apertures being five in number, the ends of each of said arc shaped plurality of apertures being adjacent to each of the longer outer edges of said conductive element.

18. The electrode of claim 15, wherein each of said plurality of apertures being a substantially straight section, and wherein each of said substantially straight sections being substantially parallel to one another, said plurality of apertures being five in number and said plurality of apertures being substantially parallel to the longer outer edges of said conductive element.

19. The electrode of claim 12, wherein:
a total edge length of said conductive element being defined as the length of said outer edge plus the length of said inner edge; and
wherein the addition of said at least one aperture to said flat conductive element causing a percentage increase of edge of greater than about 200% wherein said percentage of increase of edge being defined by the equation:
$$\% increase of edge = \frac{( total edge length ) - ( length of outer edge )}{( length of outer edge )}$$

20. The electrode of claim 19, wherein said percentage of increase of edge being between about 200% and about 300%.

21. An electrode, comprising:
a conductive element being made of aluminum, said conductive element having an outer edge, said conductive element having a plurality of apertures within said outer edge defining at least one inner edge of said conductive element, said apertures comprising:
a first pair of openings located near an end of said conductive element and extending through said conductive element, each of said first pair of openings having a substantially straight section and an arc shaped section contiguous with said substantially straight section;
a second pair of openings located adjacent to said first pair of openings, each of said second pair of openings having three substantially straight sections wherein one of said three substantially straight sections being substantially at a right angle to and contiguous with the other two of said three substantially straight sections;
a third pair of openings, said third pair of openings being substantially straight and each of said third pair of openings being substantially parallel with two of said straight sections of said second pair of openings, and each of said third pair of openings located between two of said straight sections of said second pair of openings;
a fourth pair of openings being substantially straight and being located adjacent to said second pair of openings, each of said fourth pair of openings being substantially parallel to two of said substantially straight sections of said second pair of openings;
a fifth opening located adjacent to said fourth pair of openings, said fifth opening being substantially T-shaped; and
a sixth pair of openings located near an end of said conductive element and adjacent to said fifth opening, each of said sixth pair of openings being arc shaped;
an electrical lead connected to said conductive element and being in electrical communication with said conductive element; and
a hydrogel layer contacting said conductive element and being in electrical communication with said conductive element, said hydrogel layer configured to allow electrical communication between said conductive element and an individual.

22. An electrode, comprising:
a conductive element being at least partially made of a conductive material, said conductive element having an outer edge, said conductive element having at least one recess within said outer edge defining at least one distinct inner edge of said conductive element, said recess extending only partially through said conductive element.

23. An electrode, comprising:
a conductive element being at least partially made of a conductive material, said conductive element having an outer edge, said conductive element having at least one protrusion within said outer edge defining at least one distinct inner edge of said conductive element.
